(19) United States
(12) Patent Application Publication
(10) Pub. No.: US 2023/0193238 A1
(43) Pub. Date: Jun. 22, 2023

(54) A METHOD FOR DETECTION OF WHOLE TRANSCRIPTOME IN SINGLE CELLS

(71) Applicant: SINGLERON (NANJING) BIOTECHNOLOGIES, LTD., Nanjing (CN)

(72) Inventor: Wenqi Zhu, Nanjing (CN)

(21) Appl. No.: 17/996,196

(22) PCT Filed: Apr. 16, 2020
(86) PCT No.: PCT/CN2020/085186

§ 371 Date: Oct. 13, 2022

(51) Int. Cl. C12N 15/10; C12Q 1/6806

(52) U.S. Cl. CPC C12N 15/10; C12Q 1/6806

(57) ABSTRACT

We provide a method to efficiently analyze coding RNA and non-coding RNA at single cell level in the present disclosure. A tag sequence is first added to the 3′ of RNA molecules in a single cell, and the tag sequence is subsequently used to capture said RNA and prime reverse transcription of the RNA to cDNA. The resulting cDNA can be amplified and analyzed. The tag sequence can be combined with a cell barcode sequence to decode the identity of single cells, so that a plurality of single cells can be analyzed in parallel.

### TECHNICAL FIELD

The present disclosure is a novel method for detecting whole transcriptome at single cell level, which involves single cell analysis of whole transcriptome, and in particular relates to high throughput detection of single cell whole transcriptome, including non-coding RNA.

### BACKGROUND

Single cell analysis measures DNA[1], RNA[2], and other cellular analyses at single cell resolution. Single cell analysis methods can effectively reveal heterogeneity within a sample and generate more comprehensive and accurate information. Recent technological advances in single cell partition, barcoding, and high-throughput sequencing make it feasible to examine sequences and expression profiles of genes from thousands of single cells in parallel[3]. Such high-throughput single cell sequencing techniques can be used to decipher complex biological systems. Currently, the most commonly used high-throughput single cell sequencing method is single cell mRNA sequencing, where the 3′ of the mRNA in each individual cell is quantatively detected by sequencing. Expression profiles of mRNA in single cells can be then used to annotate different cell types in a sample, and also to discover gene and pathway characteristics in each cells. The data and insights generated by single cell mRNA sequencing greatly enrich knowledge in diverse fields such as cancer[4], neurology[5], and immunology[6], and facilitate improvements in diagnosis and treatment of diseases.

However, most current single cell mRNA sequencing methods are dependent on capturing RNA by hybridization of the 3′ poly-A tail of RNA with oligonucleotides with an oligo-dT stretch[7,8]. RNA species without poly-A tails cannot be detected with such methods. Non-coding RNAs (ncRNAs) are a group of transcripts that do not code for proteins. Long non-coding RNAs (lncRNAs) form a majority of the human transcriptome and play key roles in the cellular and physiological functions, such as chromatin dynamics, gene expression, cell growth and differentiation [9]. Whole genome association studies (GWASs) of tumor samples have demonstrated that a large number of lncRNAs are associated with a variety of cancers. Changes in lncRNA expression and their mutations promote tumor occurrence and metastasis, and different lncRNAs may exhibit tumor inhibition and promotion functions[10]. Due to their tissue-specific expression characteristics and relevance in oncology, lncRNAs can be used as new biomarkers and targets for the treatment of cancer.

Micro RNAs (miRNAs) are small non-coding RNAs approximately 20 to 22 nucleotides long, which play very important roles in the regulation of target genes by binding to complementary regions of mRNAs to repress their translation or regulate their degradation [11]. This regulation appears to be involved in many fundamental cellular processes, including development, differentiation, proliferation, stress response, metabolism, apoptosis and secretion [12]. Other ncRNA species, such as snoRNA and circle RNA, have all be implicated in various aspects of the cellular functions.

The conventional methods of non-coding RNA expression analysis start by extracting total RNA from samples and then analyze total RNA, or ribosomal RNA—depleted RNA, with sequencing, microarray, or PCR[13,14]. The expression level of ncRNAs in bulk sample is an average of that in all cell types in the sample, which can mask cell-specific ncRNA expression patterns that are functionally relevant. While mRNA can be regularly detected at single cell level by methods such as SMART-seq, such methods generally start with capturing mRNA molecules through their 3′ poly-A tails with an oligo-dT RT primer[15]. Most ncRNA molecules do not have poly-A tails and cannot be captured this way at single cell level.

Some currently methods can capture whole transcriptome in single cells. However, each of these methods has it own drawbacks.

SUPeR-seq is one of such methods. This method replaces commonly used oligo-dT primers with random primers with anchor sequences, and can simultaneously capture RNAs with and without polyA tails. This method uses modified cell lysis and RT conditions to avoid capturing ribosomal RNA (rRNA), which can be about 90% of total RNA. Since cellular compositions can be different among different cell types, it remains to be tested whether this method can efficiently minimize rRNA capture in different cell types [16].

Another method, RamDA-seq, uses short NSRs (not-so-random primers) to capture and reverse transcribe RNAs while excluding rRNA. Although this method can be used to detect ncRNA, the design of the NSRs as short oligonucleotides makes it difficult to add cell barcode sequences, making it unsuitable for detecting ncRNA in multiple single cells in parallel[17].

### SUMMARY

In the present disclosure, we first extend the 3′ of the ncRNA with a stretch of oligonucleotides with specific sequences (“tag”). The tag can be added to the 3′ of the ncRNA by enzymatic or chemical approaches. The RT primer can be designed in such a way that it can bind to and capture ncRNA through the added tag sequence. Optionally, the RT primer can be combined with a oligonucleotide sequence that can act as cell barcode ad distinguish each single cell from other cells, so that thousands or more of single cells can be analyzed in parallel. This method can also be used in combination with a microfluidic system where each cells in a sample can be partitioned to individual micro-chambers. Single cells can be lyzed in the micro-chambers; tag sequence can then be added to enable capture of ncRNA with a tag-specific primer.

### BRIEF DESCRIPTION OF DRAWINGS

FIG. 1: Schematic diagram of the present disclosure.

FIG. 2: Schematic diagram of the embodiment of the present disclosure where Poly (A) Polymerase is used to add a polyA tail to the ncRNA.

FIG. 3 shows the percentage of UMI.

### DETAILED DESCRIPTION

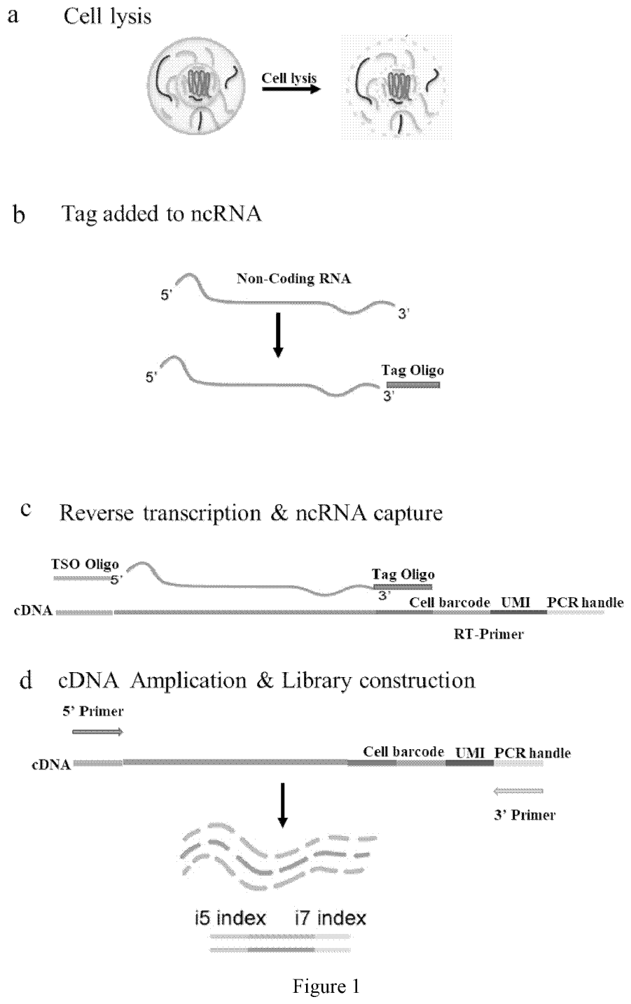

To overcome the drawbacks of the current single cell ncRNA analysis methods, we use a ncRNA-tagging method to add specific tag sequences to the ncRNA. The tag sequence can then be used to capture ncRNA molecules, and/or as priming site for RT reactions and amplification reactions (FIG. 1).

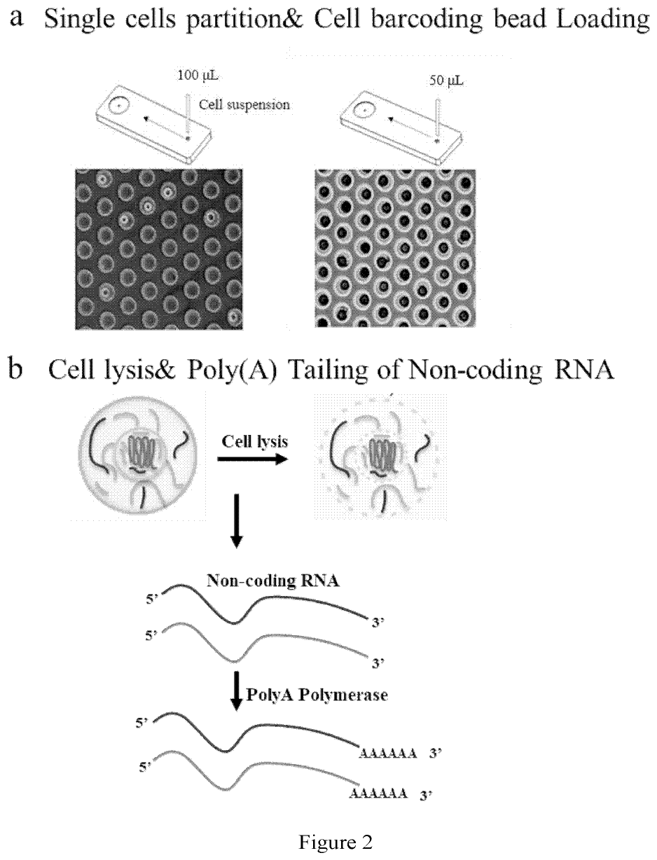

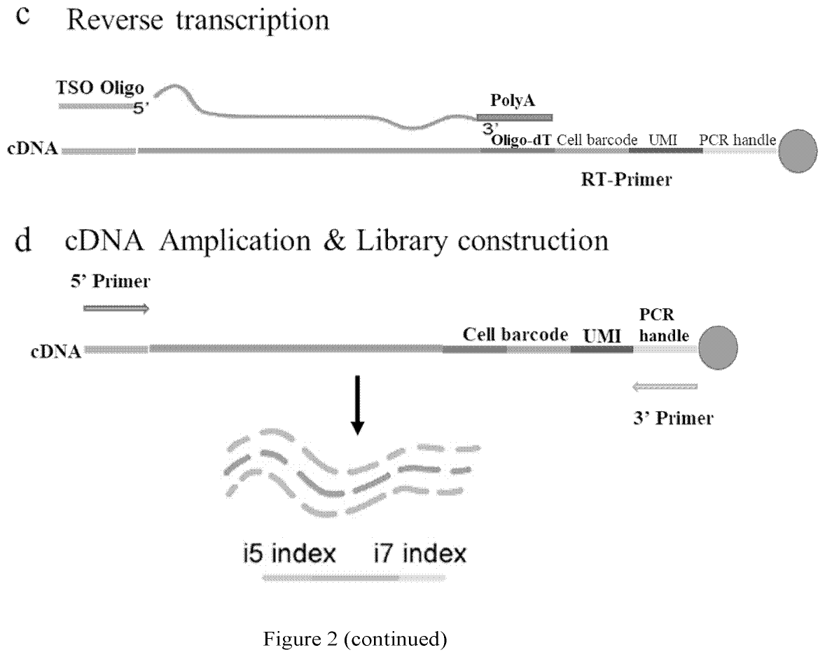

One embodiment of the present disclosure is to use Poly (A) polymerase to add a polyA tail to the ncRNA. Afterwards, oligo-dT can be used to capture and reverse transcribe both mRNA and ncRNA with newly added polyA tails. The resulting cDNA can be amplified by PCR if a template switching oligo is introduced during the RT process. With unique cell barcodes in conjunction with the oligo-dT sequence, cDNA molecules from the same single cell can be labeled and a group of single cells can be processed in parallel, enabling high-throughput single cell analysis (FIG. 2).

GEXSCOPE Single Cell RNAseq Library Construction kit (Singleron Biotechnologies) was used to demonstrate the technical feasibility and the utility of the present disclosure in massively parallel single cell ncRNA sequencing. The experiment was conducted according to manufacture's instructions with modifications described below.

Briefly, single cell suspension of K562 cells was loaded onto the microchip to partition single cells into individual wells on the chip. Four samples were prepared: two were processed with standard GEXSCOPE protocol for single cell mRNA sequencing (“control”), two were processed with modified protocol to get ncRNA reads (“nc”). Cell barcoding magnetic beads were then loaded to the microchip and washed. Each cell-barcoding magnetic bead contains oligos with a unique cell barcode sequence combined with oligo-dT on the surface. Each oligo on the bead also has a unique molecule index sequence (UMI); the number of UMIs detected in the sequence can be used to accurately quantify different RNA molecules. Only one bead can fall into each well on the microchip based on the diameters of the beads and well (about 30 um and 40 um, respectively). Instead of the lysis buffer contained in the GEXS COPE kit, the following reaction mixture was use to lyse cells and add polyA tails to the ncRNA molecules. E. coli Poly(A) Polymerase and 10×E. coli Poly(A) Polymerase Reaction Buffe are both from New England Biolabs (NEB).

| Components | Volume/Reaction (ul) |
|---|---|
| 10× E. coli Poly(A) Polymerase Reaction Buffer | 10 |
| ATP (10 mM) | 10 |
| E. coli Poly(A) Polymerase | 5 |
| 10% Triton | 2 |
| RNA inhibitor | 2.5 |
| Dnase/Rnase-Free Water | 70.5 |
| Total | 100 ul |

100 ul reaction mixture was loaded into the chip and let incubate on ice for 10 minutes to lyse cells. After the cells are lysed, the microchip was incubated at 37° C. for 30 minutes so that PolyA tails can be added to the 3′ end of RNA. After being cooled down at room temperature for 30 minutes, the magnetic beads, together with captured RNAs, were taken out of the microchip and subject to RT, template switching, cDNA amplification, and sequencing library construction using reagents from the GEXSCOPE kit and following manufacturer's instructions. The resulting single cell RNAseq library was sequenced on Illumina NovaSeq with PE150 mode and analyzed with scopeTools bioinformatics workflow (Singleron Biotechnologies).

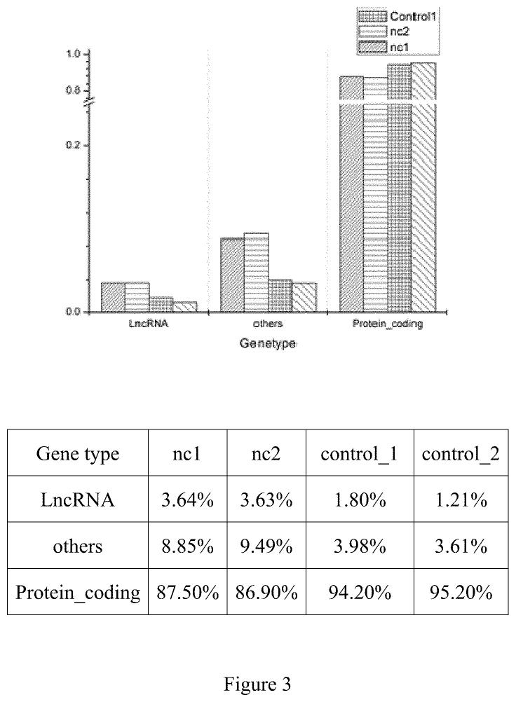

As shown in FIG. 3, the percentage of the UMIs corresponding to ncRNA in total UMIs increased more than 100%, from an average of 1.5% to 3.6%. The significantly increased percentage of ncRNA UMIs proves the principle of the present disclosure. Furthermore, the percentage of rRNA UMIs remains relatively low at (0.9%, 0.6%).

### REFERENCES

[1] Neu K E, Tang Q, Wilson P C, et al. Single-Cell Genomics: Approaches and Utility in Immunology[1]. Trends in Immunology, 2017, 38(2):140-149.
[2] Byungjin H, Hyun L J, Duhee B. Single-cell RNA sequencing technologies and bioinformatics pipelines[1]. Experimental & Molecular Medicine, 2018, 50(8):96.
[3] Klein A, Mazutis L, Akartuna I, et al. Droplet Barcoding for Single-Cell Transcriptomics Applied to Embryonic Stem Cells[J]. Cell, 2015, 161(5):1187-1201.
[4] Baslan T, Hicks J. Unravelling biology and shifting paradigms in cancer with single-cell sequencing[J]. Nature reviews. Cancer, 2017, 17(9):557-569.
[5] Ofengeim D, Giagtzoglou N, Huh D, et al. Single-Cell RNA Sequencing: Unraveling the Brain One Cell at a Time[J]. Trends in Molecular Medicine, 2017, 23(6).
[6] Papalexi E, Satija R. Single-cell RNA sequencing to explore immune cell heterogeneity[J]. Nature Reviews Immunology, 2017.
[7] Hashimshony, T., Wagner, F., Sher, N. & Yanai, I. CEL-Seq: single-cell RNA-Seq by multiplexed linear amplification. Cell Rep. 2, 666-673 (2012).
[8] Ziegenhain C, Vieth B, Parekh S, et al. Comparative Analysis of Single-Cell RNA Sequencing Methods[J]. Molecular Cell, 2017, 65(4):631-643.e4.
[9] Wu T, Du Y. LncRNAs: From Basic Research to Medical Application[J]. International Journal of Biological Sciences, 2017, 13(3):295-307.
[10] Xiaoxia Ren. Genome-wide analysis reveals the emerging roles of long non-coding RNAs in cancer. Oncol Lett. 2020 January; 19(1): 588-594.
[11] Griffiths-Jones S, Grocock R J, van Dongen S et al.miRBase: microRNA sequences, targets and gene nomenclature. Nucleic Acids Res 2006, 34:D140-4.
[12] Wijnhoven B P, Michael M Z, Watson D I. MicroRNAs and cancer. Br J Surg 2007,
[13] Nicole M White, Christopher R Cabanski, Jessica M Silva-Fisher, et al. Transcriptome sequencing reveals altered long intergenic non-coding RNAs in lung cancer[J]. Genome Biology, 15(8).
[14] Lopez J P, Diallo A, Cruceanu C, et al. Biomarker discovery: Quantification of microRNAs and other small non-coding RNAs using next generation sequencing[J]. Bmc Medical Genomics, 2015, 8(1):35.
[15] Picelli, Simone, Bjrklund, et al. Smart-seq2 for sensitive full-length transcriptome profiling in single cells[J]. Nature Methods, 2013, 10(11):1096-1098.
[16] Fan, X., Zhang, X., Wu, X. et al. Single-cell RNA-seq transcriptome analysis of linear and circular RNAs in mouse preimplantation embryos. Genome Biol 16, 148 (2015). https://doi.org/10.1186/s13059-015-0706-1

1. A method for analyzing whole transcriptome, including coding and non-coding RNA, at single cell level, wherein said method comprising:
a) add a specific tag sequence on the 3′ of RNA;
b) capture the tagged RNA with a primer that recognize the tag sequence;
c) reverse transcribe the tagged RNA to cDNA;
d) amplify cDNA;
e) analyze amplified cDNA.

2. The method of claim 1, wherein the RNA is non-coding RNA.

3. The method of claim 1, wherein the primer sequence comprises a sequence that acts as cell barcode that identifies each single cells; a specific sequence that can be used to prime the reverse transcription of the tagged RNA; and a sequence that can be used for amplification of the cDNA.

4. The method of claim 1, wherein the primer sequence comprise a unique molecular index (UMI) sequence that can be used to quantify cDNA.

5. The method of claim 1, wherein the tag sequence is added by using an enzyme.

6. The method of claim 1, wherein the tag sequence is added chemically.

7. The method of claim 5, wherein the enzyme is a Poly(A) Polymerase, to add a stretch of A to the 3′ of RNA.

8. The method of claim 5, wherein the enzyme is a terminal transferase, to add specific nucleotide sequence to the 3′ of RNA.

9. The method of claim 5, wherein the enzyme is a ligase, to add specific sequence to the 3′ of RNA.

10. The method of claim 1, wherein the analysis method is sequencing.

11. A product or kit that includes reagents needed to enable the process as described in claim 1.
